(19) United States
(12) Patent Application Publication
(10) Pub. No.: US 2016/0106534 A1
(43) Pub. Date: Apr. 21, 2016

(54) METHODS AND INTRAOCULAR LENS FOR COUNTERACTING ASTIGMATISM ABERRATIONS

(71) Applicants: Charles DEBOER, Sierra Madre, CA (US); Yu-Chong TAI, Pasadena, CA (US); Mark S. HUMAYUN, Glendale, CA (US); Wendian SHI, Monrovia, CA (US)

(72) Inventors: Charles DEBOER, Sierra Madre, CA (US); Yu-Chong TAI, Pasadena, CA (US); Mark S. HUMAYUN, Glendale, CA (US); Wendian SHI, Monrovia, CA (US)

(21) Appl. No.: 14/883,199

(22) Filed: Oct. 14, 2015

Related U.S. Application Data
(60) Provisional application No. 62/064,303, filed on Oct. 15, 2014.

(51) Int. Cl. A61F 2/16

(52) U.S. Cl. CPC A61F 2/1645; A61F 2230/0071

(57) ABSTRACT

In various embodiments, toric elements shape at least a portion of an outer shell of an implantable intraocular lens to counteract astigmatism aberrations.

### RELATED APPLICATION

This application claims the benefit of and priority to U.S. Provisional Patent Application No. 62/064,303, filed Oct. 15, 2014, the entire disclosure of which is hereby incorporated herein by reference.

### TECHNICAL FIELD

In various embodiments, the present invention relates generally to implantable intraocular lenses and, more specifically, to accommodating intraocular lenses implanted in patients' eyes.

### BACKGROUND

The crystalline lens of the human eye refracts and focuses light onto the retina. Normally the lens is clear, but it can become opaque (i.e., when developing a cataract) due to aging, trauma, inflammation, metabolic or nutritional disorders, or radiation. While some lens opacities are small and require no treatment, others may be large enough to block significant fractions of light and obstruct vision.

Conventionally, cataract treatments involve surgically removing the opaque lens matrix from the lens capsule using, for example, manual extraction and aspiration, phacoemulsification, and/or application of a femtosecond laser through a corneal incision. An artificial intraocular lens (IOL, or simply “lens”) may then be implanted in the lens capsule bag to replace the crystalline lens (see, e.g., U.S. patent application Ser. No. 14/058,634, filed Oct. 21, 2013, the entire disclosure of which is incorporated by reference herein).

Generally, IOLs are made of a foldable, optically transparent polymeric material, such as silicone or acrylic, for minimizing the incision size and required stitches and, as a result, the patient's recovery time. The most commonly used IOLs are single-element lenses (or monofocal IOLs, non-accommodating IOLs, or non-focusing IOLs) that provide a single focal distance for distance vision; the selected focal length typically affords fairly good distance vision. However, because the focal distance is not adjustable following implantation of the IOL, patients with implanted monofocal IOLs can no longer focus on objects at a close distance (e.g., less than twenty-five centimeters); this results in poor visual acuity at close distances. To negate this disadvantage, multifocal IOLs are used to provide dual foci at both near and far distances. However, due to the optical design of such lenses, patients with implanted multifocal IOLs suffer from a loss of vision sharpness (e.g., blurred vision). In addition, patients may experience visual disturbances, such as halos or glare, because of the simultaneous focus at two distances.

Recently, accommodating intraocular lenses (AIOLs) have been developed to provide adjustable focal distances (or accommodations) relying on the natural focusing ability of the eye (e.g., using contractions of ciliary muscles). Conventional AIOLs include, for example, a single optic that translates its position along the visual axis of the eye, dual optics that change the distance between two lenses, and curvature-changing lenses that change their curvatures to adjust the focus power. These designs, however, tend to be too complex to be practical to construct and/or have achieved only limited success (e.g., providing a focusing power of only 1-2 diopters).

Most IOLs are made of single piece of hard material, although some newer IOLs have a two-lens design, and lenses filled with clear fluid have also been utilized. Most current IOLs are prefabricated for their lens power and then placed in the eye, but again, a few designs involve intraocular filling of the liquid in the lens at the time of initial surgery or possibly at a subsequent time (e.g., for adjustment or should the liquid become opacified, or even simply to exchange the liquid in the lens for a liquid of different properties (e.g., optical, viscosity, color)). A liquid-filled bag that provides accommodation—made from, for example, an elastic, biocompatible polymer—results in numerous benefits and advantages, e.g., the ability to adjust the lens following implantation; to customize the lens to the needs of each patient; to accommodate vision; sharper vision over a wide range of distances; and reduction of visual side effects such as glares and halos. See, e.g., U.S. Pat. No. 8,771,347, and U.S. patent application Ser. No. 13/473,012, filed May 16, 2012, the entire disclosure of each of which is hereby incorporated by reference.

Additionally, IOL implantations may cause post-surgical complications. For example, when the crystalline lens is removed through a small incision in the anterior part of the lens capsule, the posterior side of the capsule is typically left intact to prevent vitreous humor from entering the anterior chamber of the eye. The intact posterior lens capsule, however, may develop a haze, known as posterior capsular opacification (PCO), which results in blurry vision. PCO is caused by the growth and migration of lens epithelial cells on the lens capsule, which frequently remain present following cataract surgery and represent one of the most common post-surgical complications of IOL implantation. Although laser energy (from, e.g., a neodymium-doped yttrium aluminum garnet (Nd:YAG) laser) may be utilized to open an aperture in the posterior lens capsule to remove the opacity of the capsule, and thereby restore vision, this treatment requires an extra procedure and poses an additional risk of damaging the implanted IOLs. Various treatments for PCO are disclosed in U.S. patent application Ser. No. 14/867,054, filed on Sep. 28, 2015, the entire disclosure of which is incorporated by reference herein.

Despite IOLs successfully addressing various challenges associated with eye lens replacement, it often remains difficult to fully correct astigmatism in patients afflicted therewith. Consequently, there is a need for IOLs that provide a high degree of accommodation and appropriate focusing power, and which may be shaped, before and/or after implantation, to correct astigmatism.

### SUMMARY

In accordance with various embodiments, the shape of the outer shell of an AIOL is engineered, via one or more toric elements, to counteract optical aberrations and astigmatism, thereby functioning as a toric lens. (As used herein, the term “toric lens” connotes a lens having different optical power and focal length along at least two different orientations, e.g., two orientations perpendicular to each other. Generally toric lenses have surfaces that are non-spherical, e.g., elliptical.) The AIOL is typically a fluid-filled (e.g., liquid-filled) lens, the accommodation and/or other optical properties of which are at least partially determined by the amount and/or type of fluid fill material. The toric element(s) may include or consist essentially of, for example, a solid or hollow ring shaped to constrain the outer shell of the AIOL into the desired toric shape when the AIOL is at least partially filled with fluid. (As used herein, the term “ring” may refer to a single unbroken ring or a set of two or more discrete ring portions (which may have gaps therebetween) that collectively define a ring shape.) In other embodiments, the toric element(s) include or consist essentially of one or more of the fill valves for filling the AIOL with fluid. The fill valves may be shaped and/or located on the lens in positions that preferentially shape the lens upon filling thereof. In other embodiments, the toric element(s) are regions of the outer shell itself, and such regions may have a different thickness or one or more different mechanical properties (e.g., stiffness) compared with other parts of the outer shell. Such shell regions may even be coated with a material (e.g., parylene) different from that of other portions of the outer shell.

In various embodiments, the AIOL includes the outer IOL shell, at least one internal optic enclosed therein, and a medium in the space between the internal optic and IOL shell to increase the accommodation and generate an appropriate focusing power. More specifically, the accommodation of the AIOL may be effectively altered or customized by varying the shape of the outer IOL shell, the shape and refractive index of the internal lens, and/or the volume and refractive index of the filling medium. In various embodiments, the shape of the outer IOL shell is identical for all AIOLs in order to reduce the manufacturing cost, while the properties of the internal optic and filling medium are varied to adjust the accommodation and focusing power to suit a particular patient. In addition, the shape of the outer IOL shell may be designed to accommodate the geometry of the eye (e.g., to fit within the lens capsule), thereby providing implantation comfort to the patients. Further, the surface of the AIOL may be modified to reduce or substantially eliminate the growth and migration of lens epithelial cells on the lens capsule, thereby avoiding post-surgical complications.

In some embodiments, the internal optic is a diverging optic that reduces the optical power attributable to the outer IOL shell. As a result, the accommodation of the AIOL may be beneficially increased by the outer IOL shell while the focusing power thereof is reduced to an appropriate level. In one implementation, more than one internal optic is incorporated in the AIOL; this further improves accommodation by dynamically adjusting the properties (e.g., refractive index) of each internal optic and the distances therebetween. In addition, the internal optic(s) may be integrated with the wall of the outer IOL shell to increase the thickness of the AIOL at the integrated portion(s); the thicker wall may resist the friction accompanying the AIOL insertion, thereby reducing damage to the thin portions of the AIOL walls. Because the AIOL may include two internal optics integrated with the anterior and posterior surfaces of the AIOL (resulting in thick portions on both anterior and posterior walls), the AIOL may be easily implanted into the eye via an aperture on either side of the anterior or posterior lens capsule.

Various embodiments of the invention may be customized utilizing systems and techniques described in U.S. patent application Ser. No. 14/058,634, filed on Oct. 21, 2013, the entire disclosure of which is incorporated by reference herein.

As used herein, the term “accommodation” refers both to the eye's ability to change its optical power to focus on an object at various distances and to the capacity of a lens in accordance herewith to change its optical properties in response to the eye's accommodation mechanism. In addition, the terms “optical power,” “focusing power,” “lens power,” and “refractive power” are used herein interchangeably. Optical properties that may be altered by the lens design include, without limitation, aberrations, accommodation, and/or refractive power of the lens as well as the cumulative outcome produced by one or more of the foregoing.

In an aspect, embodiments of the invention feature an implantable intraocular lens for counteracting an astigmatism aberration of a patient's eye. The lens includes or consists essentially of an outer shell defining therewithin an internal space at least partially fillable with a first fluid and a toric element disposed on or defined by the outer shell. When the outer shell is at least partially filled with the first fluid, the toric element shapes at least a portion of the outer shell to counteract the astigmatism aberration.

Embodiments of the invention may include one or more of the following in any of a variety of combinations. The toric element may include or consist essentially of a ring disposed on the outer shell. The ring may be substantially elliptical. The ring may be hollow and at least partially fillable with a second fluid. The second fluid may include, consist essentially of, or consist of the same fluid as the first fluid. The first and second fluid may have different optical clarities (e.g., opacities or transmissivities), different refractive indices, different densities, and/or different viscosities. The ring may include, consist essentially of, or consist of two or more discontinuous ring portions. The toric element may include, consist essentially of, or consist of one or more valves extending through the outer shell for selective passage therethrough of the first fluid. The one or more valves may be disposed concentrically around an optical zone of the lens (e.g., at least 3 mm from the central optical axis of the lens). At least a first portion of the outer shell may have a first thickness. The toric element may include, consist essentially of, or consist of one or more second portions of the outer shell having a second thickness greater than the first thickness. A thickness transition between a first portion of the outer shell and a second portion of the outer shell may be substantially continuous. A thickness transition between a first portion of the outer shell and a second portion of the outer shell may be abrupt (i.e., stepwise). At least a first portion of the outer shell may have a first stiffness. The toric element may include, consist essentially of, or consist of one or more second portions of the outer shell having a second stiffness greater than the first stiffness. The toric element may include, consist essentially of, or consist of one or more portions of the outer shell having a coating disposed thereon. The coating may include, consist essentially of, or consist of parylene. One or more alignment markings may be disposed on the outer shell. A degree of filing of the outer shell with the first fluid may determine, at least in part, one or more optical properties of the lens (e.g., a degree of accommodation, a focal length, etc.).

In some embodiments, the outer shell is accommodating. For example, the outer shell may adjust over a flexible range astigmatism that includes the astigmatism on which lens modeling is based. In other embodiments, the outer shell is non-accommodating, e.g., the lens may be made taut by overfilling or may be filled with a viscous material preventing accommodation; in such cases, the lens aberration may be adjustable to customize the lens for a particular patient. In some embodiments, the lens may further comprise an internal optic at least partially within the outer shell; the internal optic may comprise, consist of, or consist essentially of one or more toric elements. The outer shell may include one or more multi-focal elements, one or more diffractive elements, and/or one or more apodized elements.

In another aspect, embodiments of the invention feature a method for counteracting an astigmatism aberration of a patient's eye. An intraocular lens is implanted within the patient's eye. The intraocular lens has an outer shell and a toric element disposed on or defined by the outer shell. The lens is at least partially filled with a first fluid, whereby the toric element shapes at least a portion of the outer shell to counteract the astigmatism aberration.

Embodiments of the invention may include one or more of the following in any of a variety of combinations. The toric element may include, consist essentially of, or consist of a ring disposed on the outer shell. At least a portion of the ring may be at least partially filled with a second fluid. The toric element may include, consist essentially of, or consist of one or more valves. The lens may be at least partially filed with the first fluid via one or more of the valves. The lens may be aligned within the patient's eye via one or more alignment markings disposed on the outer shell.

Embodiments of the invention may include one or more of the following in any of a variety of combinations.

These and other objects, along with advantages and features of the present invention herein disclosed, will become more apparent through reference to the following description, the accompanying drawings, and the claims. Furthermore, it is to be understood that the features of the various embodiments described herein are not mutually exclusive and may exist in various combinations and permutations. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, routines, steps, or characteristics may be combined in any suitable manner in one or more examples of the technology. As used herein, the terms “approximately” and “substantially” mean ±10%, and in some embodiments, ±5%. The term “consists essentially of” means excluding other materials that contribute to function, unless otherwise defined herein. Nonetheless, such other materials may be present, collectively or individually, in trace amounts.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIGS. 1A and 1B depict sectional side views, respectively, of a human eye and an accommodating intraocular lens (AIOL) in accordance with various embodiments of the invention;

FIGS. 2A and 2B schematically depict an AIOL in accordance with various embodiments of the invention;

FIGS. 3A-3C schematically depict side views of various AIOLs in accordance with various embodiments of the invention;

FIG. 4 depicts a three-lens system in accordance with various embodiments of the invention;

FIG. 5 depicts a three-lens AIOL in accordance with various embodiments of the invention;

FIGS. 6A and 6B illustrate an AIOL in an accommodated state and in a natural state without accommodation, respectively, after implantation in the lens capsule of an eye in accordance with various embodiments of the invention;

FIG. 7 schematically depicts an AIOL including an anterior capsulotomy and a posterior capsular aperture in accordance with various embodiments of the invention;

FIG. 8A is a schematic view of an AIOL having a spherical surface shape in accordance with various embodiments of the invention;

FIG. 8B is a schematic view of a toric AIOL having an oval surface shape in accordance with various embodiments of the invention;

FIG. 9A is a schematic view of an AIOL having an equator with a circular shape in accordance with various embodiments of the invention;

FIG. 9B is a schematic view of an AIOL having an equator with an elliptical shape in accordance with various embodiments of the invention;

FIGS. 10A-10 are schematic views of an AIOL having an elliptical ring incorporated thereon in accordance with various embodiments of the invention;

FIGS. 11A-11C are schematic views of an AIOL having a hollow fillable ring incorporated thereon in accordance with various embodiments of the invention;

FIG. 12 is a schematic view of an AIOL having multiple fill valves configured to produce a toric lens shape in accordance with various embodiments of the invention; and

FIG. 13 is a schematic view of an AIOL having surface and/or thickness portions configured to produce a toric lens shape in accordance with various embodiments of the invention.

### DETAILED DESCRIPTION

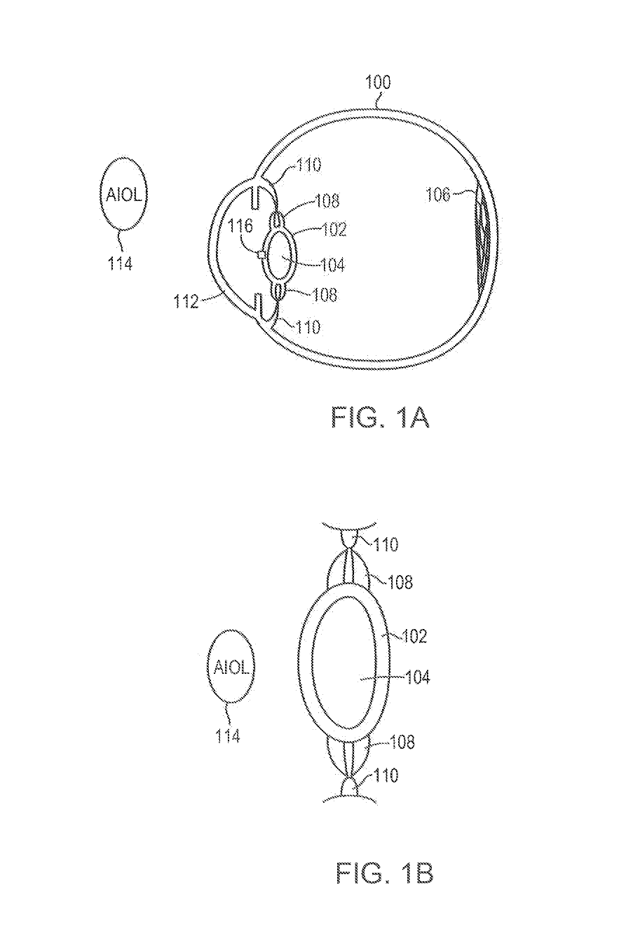

FIGS. 1A and 1B illustrate the structure and operation of a human eye 100. The eye 100 has a lens capsule 102 with a crystalline lens 104 that focuses light onto the retina 106; the lens capsule 102 is joined by ligament fibers 108 around its circumference to ciliary muscles 110, which are further attached to the inner surface of the eye 100. In various embodiments of the present invention, during cataract surgery, lens 104 is removed from the lens capsule 102 using, for example, phacoemulsification and/or a femtosecond laser through a small incision in the periphery of the patient's cornea 112. An AIOL 114 as further described below is inserted through a small incision on the anterior capsule portion 116 into the lens capsule 102. The surgeon then ensures that the AIOL 114 is deployed and placed correctly and that there are no tears in the capsule 102.

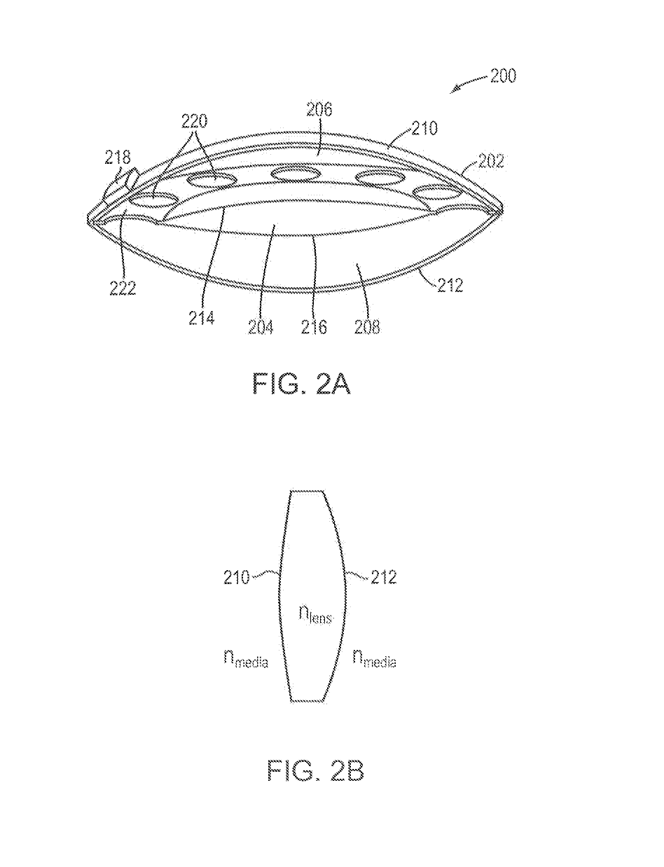

Referring to FIG. 2A, in various embodiments, the AIOL 200 includes an outer intraocular lens 202 enclosing an internal optic 204, a medium-filled anterior compartment 206 and a posterior compartment 208 created between the intraocular lens 202 and the internal optic 204. The internal optic 204 is not necessarily spherical and may be of any shape to obtain the desired outcome. The internal optic may further counteract a patient-specific aberration (e.g., to induce a toric optical outcome). Additionally, the internal optic 204 may be positioned at the equator of the AIOL 200 or at another location within the AIOL 200. The focusing power of the AIOL 200 may be varied by adjusting the shape (or curvature) of the outer IOL 202, the shape and/or refractive index of the internal optic 204, and/or the volume and/or refractive index of the filling medium.

During eye accommodation, ciliary muscles 110 release tension applied to the lens capsule 102, which results in a shape change of the lens capsule 102 and the AIOL 200 implanted therein. For example, the anterior surface 210 or posterior surface 212 of the AIOL 200 may undergo a change in radius of curvature and/or move in the anterior or posterior direction. In one embodiment, the outer IOL 202 of multiple different AIOLs 200 is molded in the same geometry (e.g., a one-size-fit-all outer IOL 202) to reduce manufacturing costs. In other embodiments, the outer IOL 202 is molded with one or more specific structures to correct for and/or controllably induce astigmatism. The eye's accommodation level is controlled by the shape and refractive index of the internal optic 204 and/or the index of the medium filling the anterior compartment 206. For example, to satisfy patients having various focusing power requirements, the AIOLs 200 may be produced with identically shaped outer IOLs 202 while incorporating different internal optics 204 and/or different filling media therein to provide different focusing powers. In some embodiments, the shape of the outer IOL 202 is designed to conform to the geometry of the eye (e.g., fitting within the lens capsule 102); this ensures the patient's comfort and the achievement of good focusing power after the AIOL implantation. Alternatively, the outer IOL 202 may be manufactured in a few different sizes that all fit within a typical patient's lens capsule or may be customized for the lens capsules of particular patients. This approach may reduce the amount of additional correction required from the internal optic and/or filling medium. In some embodiments, the outer IOL corrections negate the need for additional correction from the internal optics.

Referring to FIG. 2B, the optical power (which is typically defined as the inverse of focal length) of an AIOL having no internal optic 204 in a surrounding medium (e.g., aqueous humor) may be approximately computed using the lensmaker's formula for a thin lens:

$$Power = \frac{1}{f} = \frac{{n}_{lens} - {n}_{media}}{{n}_{media}} [ \frac{1}{{R}_{1}} - \frac{1}{{R}_{2}} ] , \qquad Eq . ( 1 )$$

where n_{media} and nₗₑₙₛ are the refractive indices of the surrounding medium and AIOL, respectively, and R₁ and R₂ are the curvatures of the anterior lens surface 210 and posterior lens surface 212 of the AIOL, respectively. If changes in the relative position of the anterior lens surface 210 and posterior lens surface 212 are negligible, the accommodation of the AIOL may be approximated as:

$$Accom = \frac{{n}_{lens} - {n}_{media}}{{n}_{media}} ⌊ \frac{1}{Δ {R}_{1}} - \frac{1}{Δ {R}_{2}} ⌋ . \qquad Eq . ( 2 )$$

Equation (2) indicates that once the refractive index of the AIOL has been determined, the accommodation of the AIOL 200 is adjustable by changing the radius of curvature of the anterior surface 210 and/or the posterior surface 212 of outer shell 202. In addition, the accommodation level may be increased by using an AIOL with a larger refractive index. For example, assuming n_{media} is roughly 1.336, varying nₗₑₙₛ from 1.4 to 1.47 results in a calculated increase in the accommodation level by a factor of approximately 2.1 (i.e., from 5 diopters to 10.5 diopters). This increase in accommodation comes at a cost, however: the overall power of the IOL increases, as indicated in Equation (1). To address this, a diverging optic may be placed in the center as described below; an internal optic with a negative power compensates for the increase in overall power.

It should be noted that although the focusing power and accommodation are described herein using the thin lens formula, a thick lens formula may be used to predict increasing focusing power and accommodation level with an increased refractive index of the AIOL.

The filling medium may include, consist essentially of, or consist of one or more refractive liquids, gels, curable polymers, or a compressible gas. In various embodiments, the AIOL 200 is implanted with compartments 206 and 208 unfilled; this enables the AIOL 200 to be easily rolled up for ease of introduction into the lens capsule 102 of an eye. After implantation, one or more media are introduced into the AIOL 200 via, for example, a refill valve 218. Upon injection, the filling medium flows from the anterior compartment 206 to the posterior compartment 208 via one or more apertures 220 on a septum 222 that supports the internal optic 204. In other embodiments, each compartment may have its own associated refill valve, thereby allowing for isolated compartments. In some embodiments, the medium is a pharmacological agent that may be released from or diffuses out of the AIOL 200 in a controlled manner (e.g., using a controller) and may be replenished using the valve 218. Accordingly, the implanted AIOL 200 may be a replacement of the crystalline lens and/or a drug pump delivering a therapeutic agent to the eye.

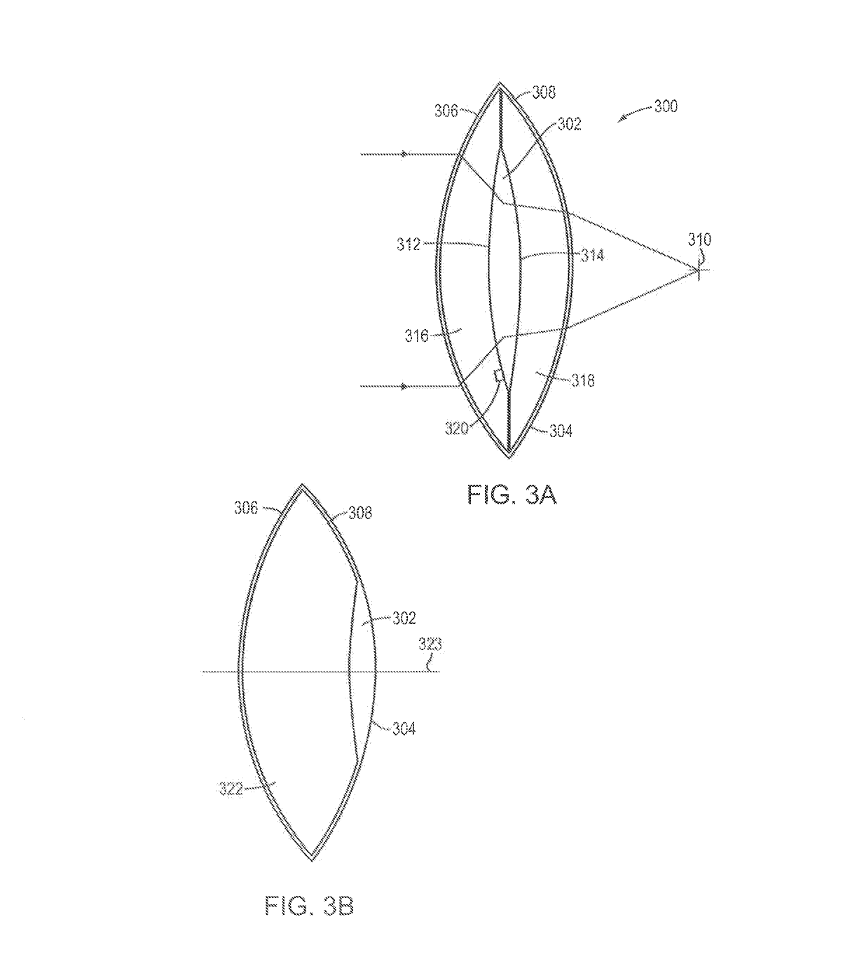

Equations (1) and (2) indicate that increasing the accommodation level using a high-index AIOL (e.g., by increasing the refractive index of the filling medium) comes at the cost of a high optical power. In fact, by increasing the accommodation level by a factor of 2.1, the optical power is also approximately increased by a factor of 2.1. Referring to FIG. 3A, in various embodiments, the internal optic of an AIOL 300 includes a diverging lens 302 to reduce the overall optical power. Because the outer IOL 304 has a converging anterior lens surface 306 and a converging posterior lens surface 308 (thereby providing a positive focusing power), embedding a diverging lens, which has a negative focusing power, reduces the overall optical power of the AIOL 300. More specifically, when collimated light emitted from a light source first enters the AIOL 300, the light is refracted inward by the anterior lens surface 306 of the AIOL 300. The light is then refracted outward by the internal diverging lens 302, then by refraction inward at the posterior lens surface 308, ultimately focusing at a position 310. The diverging lens 302 may be a convex or concave lens as long as its refractive index is selected to create a negative focusing power based on the refractive index of the surrounding medium in the anterior and posterior compartments 316, 318. Accordingly, the AIOL 300 advantageously uses the diverging lens 302 to reduce focal power while providing a high accommodation level that may be adjusted by varying the curvatures of the anterior and posterior lens surfaces 306, 308 of the AIOL 300, the curvatures of the anterior and posterior surfaces 312, 314 of the internal optic 302, and the refractive index of the internal optic 302.

The internal optic 302, which may be used to adjust accommodation and focusing power for treating emmetropia, optical aberrations, and astigmatism, may include, consist essentially of, or consist of one or more solid materials, such as acrylic, silicone, polymethyl methacrylate (PMMA), and/or parylene. Alternatively, the internal optic 302 may be fillable with fluid, in which case the refractive index of optic 302 may be tuned by changing the volume and/or components of the fluid. In one embodiment, the fluid filling the internal optic 302 repels the medium filling the anterior and posterior compartments 316, 318; this prevents interdiffusion or exchange of the fluid and medium so that each separately maintains a constant refractive index. For example, the internal optic 302 may be filled with a hydrophilic liquid, such as sugar water, while the anterior compartment and posterior compartments 316, 318 are filled with a hydrophobic medium, such as silicone oil. As a result, the silicone oil prevents the water or sugar moieties from diffusing into the anterior compartment and posterior compartments 316, 318, and the sugar water prevents the silicone oil from diffusing into the internal optic 302. In some embodiments, the internal optic 302 includes a valve 320 to permit fluid to be injected into the internal optic 302 prior to or after the AIOL implantation.

The internal optic 302 may be a separate component located within the outer IOL 304 as described above. Alternatively, with reference to FIG. 3B, the internal optic 302 may be integrated with the outer IOL 304; that is, the internal optic 302 is defined by the outer IOL 304 such that their surfaces are continuous with each other. This creates a single internal chamber 322 that may be filled with a fluid medium. Again, the accommodation of the AIOL may be adjusted by changing the volume or refractive index of the filling medium. In one embodiment, the internal optic 302 is integrated with a portion of the outer IOL 302 along the optical axis 323 thereof. During accommodation, the shape of the optic 302 may remain unchanged while its position translates along the optical axis due to the shape change of the IOL surfaces 306, 308. The internal optic 302 may have any suitable shape and may have the same or a different refractive index from that of the IOL wall and/or the filling medium in the chamber 322. In order to suit the needs of various patients, the overall accommodation and focal power of AIOL embodiments having an internal optic 302 integrated with the outer IOL 304 may be adjusted by the shape of the IOL walls 306, 308, the position translation of the internal optic 302, and/or the refractive index and volume of the filling medium in the chamber 322.

Because the thickness of the AIOL in the portion where the internal optic 302 is integrated is thicker and less flexible than other areas of the AIOL, the integrated inner optic 302 may advantageously prevent the AIOL from bulging across the capsular incision used to insert the AIOL or an incision in the posterior portion of the lens capsule. In addition, the AIOL may be rolled up with the thicker portion facing outwards; as a result, friction occurring during the AIOL insertion is exerted on the thicker portion. This reduces the risk of damaging the thin-walled areas of the AIOL during implantation.

Referring to FIG. 3C, in various embodiments, the AIOL includes two internal optics 324, 326 that may be located anywhere inside the outer IOL 304. Again, the internal optics 324, 326 may be composed solely of solid materials or filled with fluids through refill valves 328, 330 thereon. Because each internal optic may be filled with fluid individually, its refractive index is independently variable, providing an additional adjustment for the optical power and accommodation of the AIOL. Therefore, the overall accommodation and/or focusing power of the AIOL may be adjusted by various factors, including the shape of the outer IOL 304, the shapes of the internal optics 324, 326, the refractive indices of the fluids contained in the optics 324, 326, the refractive index of the medium in the chamber 332, and/or the axial distance 334 between the two optics 324, 326. In addition, this design advantageously allows the AIOL to be entirely or partially deflated during insertion and inflated following insertion (using, for example, refilling valves 328, 330, 336), thereby significantly reducing the incision size.

In one embodiment, the two internal optics 324, 326 are integrated with (i.e., form a part of) the anterior inner surface 306 and posterior surface 308 of the outer IOL 304, respectively; this AIOL may be suitable for a capsulotomy procedure on either surface (i.e., anterior or posterior surface) of the lens capsule. Again, because the thickness of the AIOL in the portions where the internal optics 324, 326 are integrated is thicker and less flexible than other areas of the AIOL, the AIOL surfaces having the integrated inner optics 324, 326 may be used to withstand the brunt of the friction occurring during the AIOL insertion. Additionally, the thicker portion of the posterior surface 308 may reduce the risk of damaging the AIOL when a YAG capsulotomy (i.e., application of laser energy to ablate a portion of the posterior lens capsule) is performed on the posterior lens capsule for treating or preventing PCO.

To negate post-surgical complications and thereby obviate the need for YAG capsulotomy, in one embodiment, the outer surface of the AIOL is modified with functional groups, such as hydroxyl, amine or amide, carboxylic acid, fluoro-modified groups, alkyl groups, parylene, and/or implanted with pharmaceuticals (such as chemotherapy agents) to kill and/or prevent movement of lens epithelial cells. In another embodiment, the outer surface of the AIOL includes an angled circumferential edge 337 to create a discontinuity thereon and/or an adhesive member 338. The discontinuity 337 and/or adhesive layer 338 prevent lens epithelial cells from migrating and causing PCO or anterior capsular opacification (ACO). In one embodiment, the circumferential edge 337 and/or adhesive layer 338 is placed outside the central optical zone (e.g., 3 mm away from the optical axis 323) of the AIOL. In addition, the circumferential edge 337 and/or adhesive member 338 may enhance the mechanical coupling between the lens capsule and AIOL, thereby increasing the accommodation thereof.

In the dual internal lens system, the effective focal length may be adjusted by changing the distance 334 between the lenses 324, 326. For example, the effective focal length, EFL, of the AIOL depicted in FIG. 3C may be defined as:

$$EFL = \frac{{f}_{1} \times {f}_{2}}{{f}_{1} + {f}_{2} - d} , \qquad Eq . ( 3 )$$

where f₁ and f₂ are the focal lengths of the first and second lenses 324, 326, respectively, and d is the distance 334 therebetween. Accordingly, changing the distance d results in a change of the effective focal length. In some embodiments, either f₁ or f₂ is negative (i.e., using a diverging lens), resulting in a smaller sum f₁+f₂ and giving the distance d a larger influence over the effective focal length. This effect is similar to using a large change of refractive index in the AIOL with a given distance d.

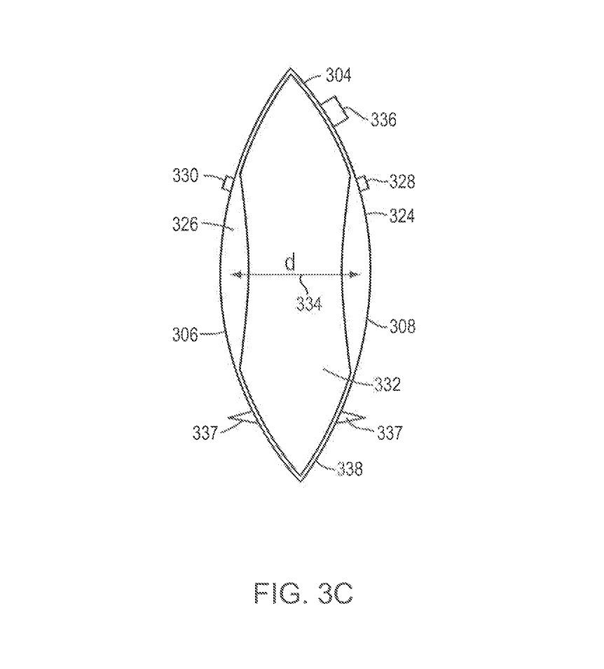

Referring to FIG. 4, in some embodiments, the AIOL 400 includes more than two internal optics. The effective focal length is highly influenced by the distances between the lenses. For example, in a three-lens system 400, the effective focal length may be expressed as:

$$EFL = \frac{( \frac{{f}_{1} \times {f}_{2}}{{f}_{1} + {f}_{2} - {d}_{1 - 2}} ) \times {f}_{3}}{( \frac{{f}_{1} \times {f}_{2}}{{f}_{1} + {f}_{2} - {d}_{1 - 2}} ) + {f}_{3} - {d}_{2 - 3}} , \qquad Eq . ( 4 )$$

where d₁₋₂ is the distance between lens 402 and lens 404; d₂₋₃ is the distance between lens 404 and lens 406; and f₁, f_{2,} and f₃ are the focal lengths of the lenses 402, 404, 406, respectively. Similar to the two-lens system, the effective focal length of the three-lens system 400 may be tuned by varying the relative distances between the three lenses 402, 404, 406 and the radius of curvature of each lens (which affects the individual focal length). Additionally, the focal length (and therefore the focal power) of the three-lens system 400 may also be adjusted by modifying the refractive index of the medium surrounding the lenses 402, 404, 406 based on equations similar to Equations (1) and (2). Accordingly, the overall optical power and accommodation of a multiple-lens system is determined by the curvature and refractive index of each lens, the relative positions therebetween, and the refractive index and volume of the filling medium. In addition, when the implanted lens system changes its position in the lens capsule, the overall focusing ability of the eye may change as well.

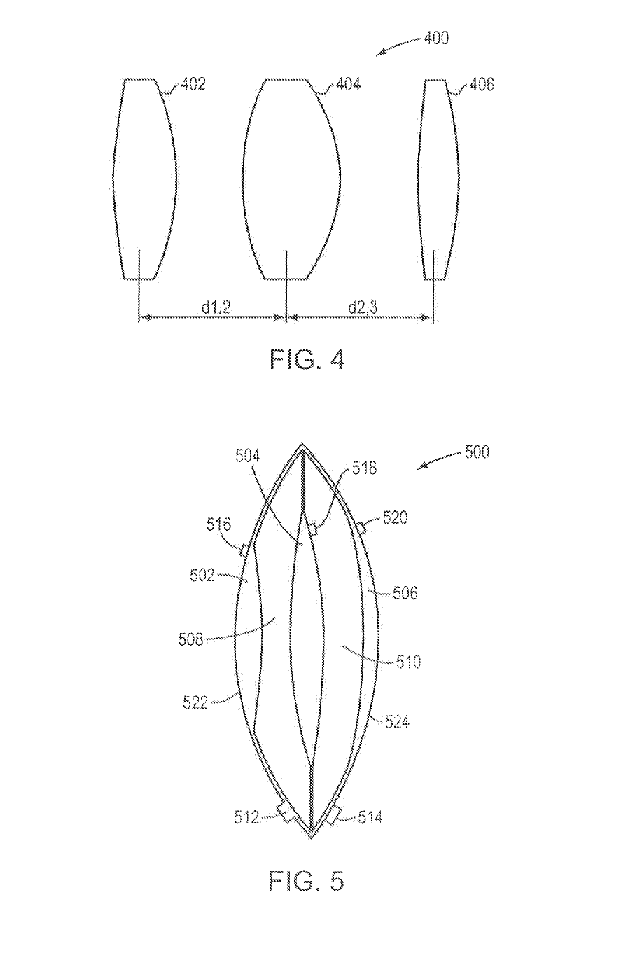

Referring to FIG. 5, in various embodiments, an AIOL 500 includes three internal optics: an anterior optic 502, a central optic 504, and a posterior optic 506. An anterior compartment 508 and a posterior compartment 510 located between the optics 502, 504, 506 may be in fluidic connection and filled with the same medium. The filling medium may include, consist essentially of, or consist of one or more of an optical fluid, a gel, a curable polymer, or a compressible fluid (e.g., a gas). In various embodiments, the AIOL 500 is inserted into the eye's lens capsule with the anterior and posterior compartments 508, 510 evacuated; this allows the AIOL 500 to be compressed and/or folded to facilitate implantation through a small surgical incision. After the AIOL 500 is implanted in the lens capsule, the anterior compartment 508 and posterior compartment 510 are filled with a filling medium via a refill valve 512. The volume and components of the filling medium are chosen to optimize the accommodation level and refractive power of the AIOL 500 after implantation. In one embodiment, the anterior compartment 508 and the posterior compartment 510 are not in fluidic connection, and the AIOL 500 includes a second refill valve 514 to access the posterior compartment 510 after implantation. Accordingly, the filling volumes of the media in the anterior and posterior compartments 508, 510 may be adjusted individually to optimize the overall accommodation of the AIOL 500 based on the needs of each patient.

Each of the internal optics 502, 504, 506 may be composed of a solid material or filled with a fluid (a liquid and/or a gas) that is injected through refill valves 516, 518, 520, respectively. The valves 516, 518, 520 allow the refractive indices of the optics 502, 504, 506 to be adjusted simultaneously or subsequently by changing the filling volume and/or components of the filling fluid(s). The fluid may include, consist essentially of, or consist of one or more of a flexible polymer, gel, liquid, or compressible fluid to allow the optics 502, 504, 506 to flex during accommodation. The fluid in the optics 502, 504, 506 and the medium filling the anterior and posterior compartments 508, 510 may have the same or different refractive indices and may repel each other to prevent diffusion therebetween. In various embodiments, one or more of the three internal optics 502, 504, 506 are aspheric to minimize optical aberration or designed in a shape to counteract astigmatism, thereby functioning as a toric lens. In addition, the internal optics 502, 504, 506 may be deflated or inflated prior to the AIOL implantation into the eye. In various embodiments, the anterior optic 502 and posterior optic 506 are integrated with (i.e., physically part of) the anterior wall 522 and posterior wall 524 of the enclosing IOL, respectively. Again, the thicker portions of the AIOL walls due to the integration of the optics may be used to resist the friction occurring during the AIOL 500 insertion.

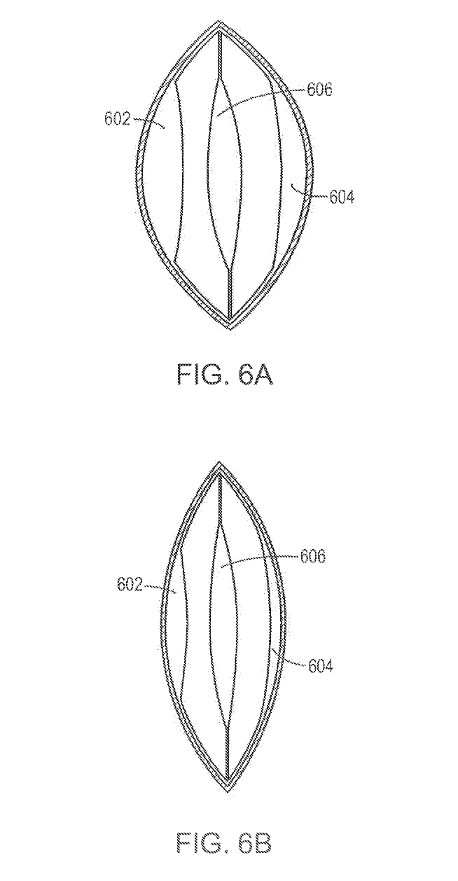

FIGS. 6A and 6B illustrate the operation of the AIOL, in accordance with various embodiments of the present invention, after implantation in the lens capsule of a patient's eye. Referring to FIG. 6A, during accommodation, the anterior optic 602 and posterior optic 604 move apart (i.e., the distance therebetween increases); as a result, the overall focusing power increases and thus allows light to be focused on an object at a close distance. The curvatures of the anterior and posterior optics 602, 604 may or may not change during accommodation. In another embodiment, the central optic 606 moves preferentially to either the anterior or posterior side of the lens during accommodation. The septum that supports the internal optic may be biased toward the anterior or posterior side of the lens. In various embodiments, the septum may include concentric corrugations to further improve the preferential movement to the anterior or posterior side of the lens during accommodation. The movements of the optics 602, 604, 606 provide accommodation adjustments. Referring to FIG. 6B, if the AIOL is in a natural state without accommodation, the anterior and posterior optics 602, 604 are closer to each other and less curved; this results in a lower overall optical power of the AIOL.

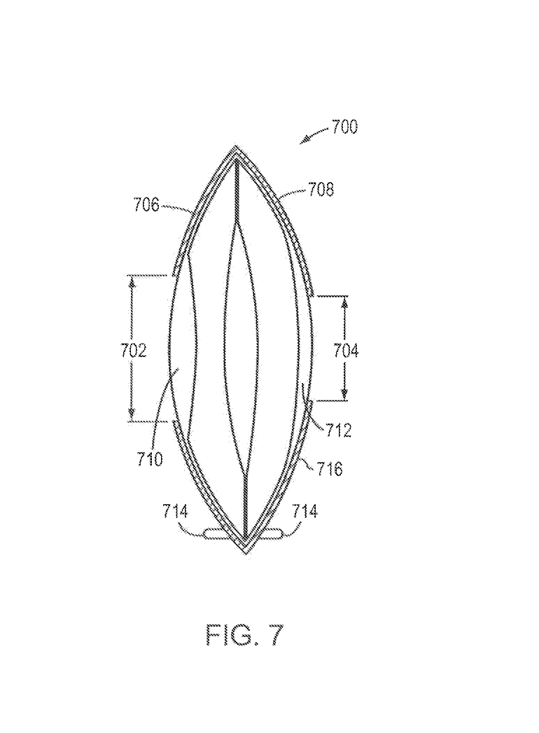

Referring to FIG. 7, in some embodiments, an AIOL 700 includes an anterior aperture (i.e., capsular capsulotomy) 702 and a posterior capsular aperture 704 on a anterior wall 706 and a posterior wall 708, respectively, to, for example, alleviate ACO and PCO complications. The anterior and/or posterior capsular apertures 702, 704 may be created using, for example, a manual instrument or a laser. Because the anterior optic 710 and posterior optic 712, placed across the anterior aperture 702 and posterior aperture 704, respectively, are relatively rigid, the optics 710, 712 provide support to the AIOL 700 during accommodation and relaxation thereof. In various embodiments, the AIOL 700 includes a discontinuous feature (e.g., a groove ring) 714 on the anterior and/or posterior surface to prevent the AIOL 700 from potentially bulging out of the capsular aperture on the lens capsule. The discontinuous feature 714 placed between the AIOL and the surrounding lens capsule may prevent lens epithelial cells from migrating from the lens capsule across to the lens optic. For example, the AIOL may be in contact with the peripheral lens capsule; a groove on the AIOL prevents lens epithelial cells from traveling to the optical center of the AIOL. In one embodiment, the outer surface of the AIOL includes an adhesive layer 716 that mechanically connects the AIOL 700 to the lens capsule; this may prevent the lens epithelial cells from proliferating or migrating as well as enhance the AIOL 700 accommodation. In addition, both anterior and posterior lens capsules may be excised (e.g., each lens capsule may have some material removed therefrom) during the AIOL implantation; this limits the proliferation and migration of the lens epithelial cells along the optical axis of the eye, thereby mitigating post-surgical complications.

Embodiments of the invention may also be utilized to correct astigmatism. There are two different types of sources of astigmatism in patients' eyes, namely astigmatism aberration in the natural lens and astigmatism aberration in the cornea. In the first case, a normal intraocular lens may be used to replace the natural lens and often eliminates the astigmatism. In the second case, however, a normal lens typically does not eliminate the astigmatism in the cornea, and therefore a toric lens is used to compensate the astigmatism aberration in the cornea and eliminate the overall astigmatism. A toric lens with designed astigmatism of proper amount and correct orientation is crucial for this application. Techniques to introduce designed astigmatism in a liquid-filled lens are described herein, enabling the liquid-filled lens to be adapted to function as a toric lens. Such techniques may be implemented on the anterior and/or posterior surfaces of the IOL.

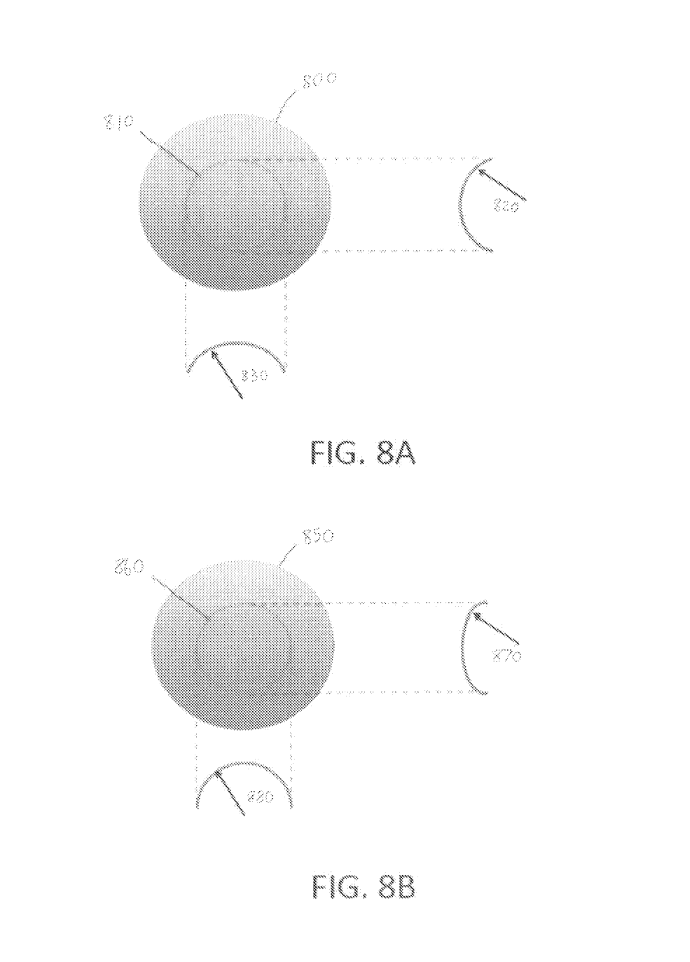

In a regular (i.e., non-toric) liquid-filled lens, the optical zone of the lens surface has a spherical profile, which exhibits the designed spherical power to match base power of the patient's eye, as shown in FIG. 8A. As shown, a liquid-filled lens 800 has an optical zone 810 in the center portion of the lens. At least in the optical zone 810, the profile of the lens 800 is spherical, i.e., radii 820, 830 along perpendicular directions are approximately equal and define circular cross-sections. In contrast, in a toric liquid-filled lens 850, surface of the lens in an optical zone 860 has an oval profile, as shown in FIG. 8B. That is, radius 870 defines an elliptical cross-section while radius 880 defines a circular cross-section. In this manner, lens 850 exhibits not only the designed spherical power to match the base power, but also the cylinder power to match and compensate the astigmatism of the patient's eye.

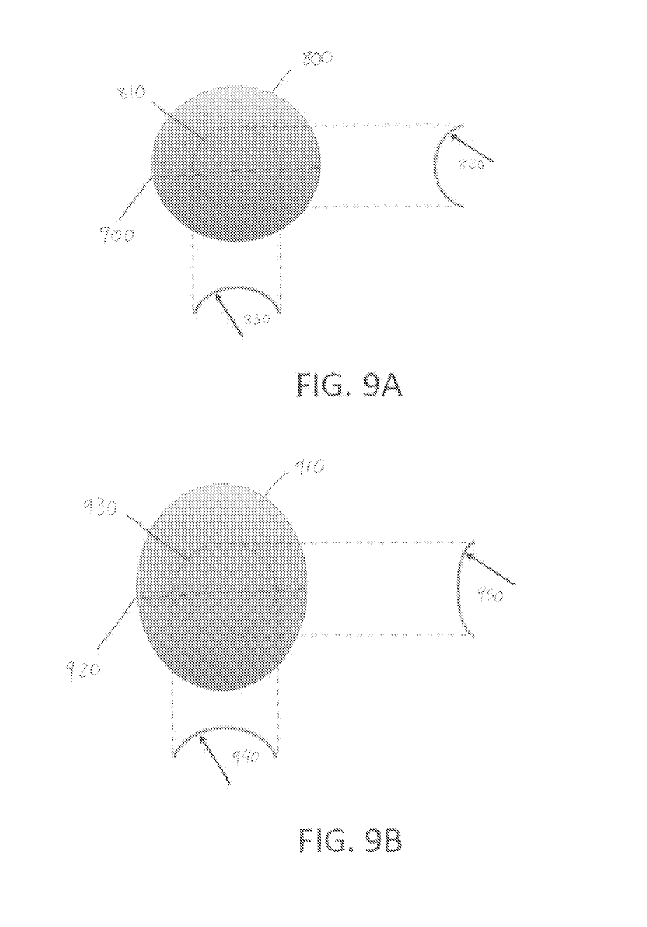

There are several ways to modify a spherical profile of the lens surface into the oval profile of FIG. 8B. One way to introduce the oval profile is to use an elliptical shape of the equator instead of the circular shape of the equator. As shown in FIG. 9A, since lens 800 has a circular-shaped equator 900, the profile of the lens surface (at least in the optical zone 810) is isotropic. Hence, when the lens 800 is filled up with liquid, the expansion of the surface membrane is also isotropic and forms a spherical shape. FIG. 9B depicts a lens 910 having an equator 920 having an elliptical shape (at least in an optical zone 930). As shown, the profile of the surface of lens 910 is non-isotropic. Specifically, after the lens 910 is filled with liquid, the lens surface along the major axis has a larger radius of curvature 940, whereas the lens surface along the minor axis has a smaller radius of curvature 950. The difference in the radius of curvature introduces a different amount of optical power along the two axes, thereby making the lens 910 toric.

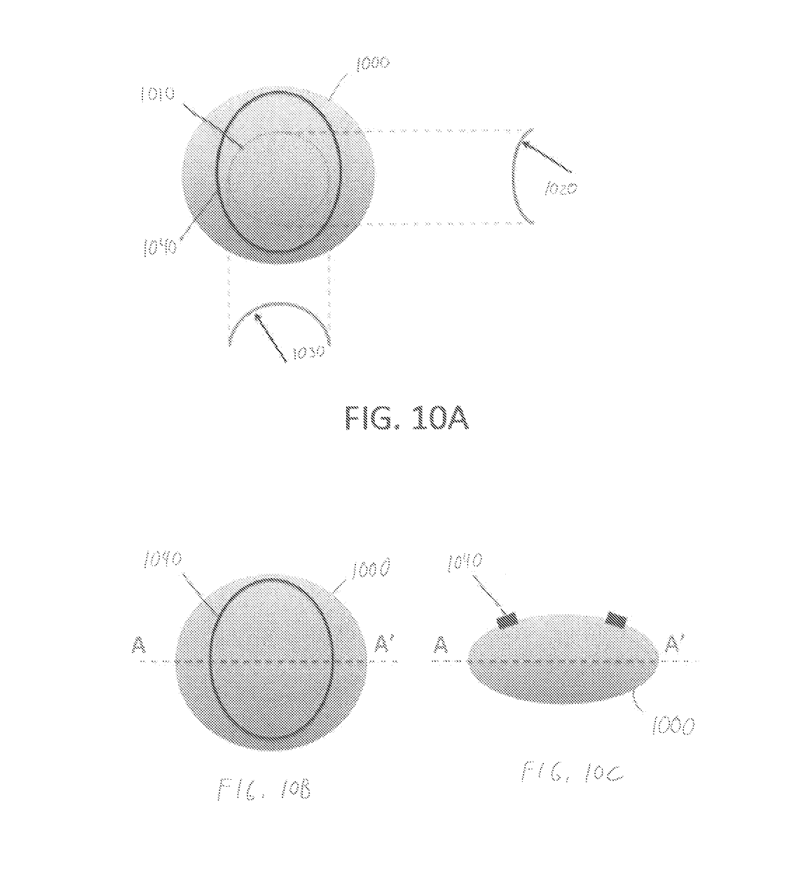

Various embodiments of the invention introduce an oval/elliptical profile to the surface of the lens by incorporating an elliptical ring on or in the lens. The incorporation of the elliptical ring allows the surface profile of the membrane in the optical zone to be altered without modifying the total profile of the lens. FIG. 10A depicts a liquid-filled lens 1000 having an optical zone 1010 (which has radii of curvature 1020, 1030 in perpendicular directions) and an elliptical ring 1040 on the lens surface. In various embodiments, the elliptical ring 1040 has a mechanical strength stiffer than the remaining portion of the lens surface membrane. FIG. 10B depicts a top view of lens 1000, and FIG. 10C depicts a cross-sectional view of lens 1000 though line A-A′. When lens 1000 is filled with liquid, the elliptical ring 1040 constrains the deformation of the lens surface membrane. Along the major axis of the elliptical ring 1040, the lens membrane deforms to have a larger radius curvature 1020, and hence a lower optical power. Along the minor axis of the elliptical ring 1040, the lens membrane deforms to have a smaller radius of curvature 1030, and hence a higher optical power. In this way, the lens surface becomes toric, and the amounts of the spherical power and the cylinder power are determined by the shape of the elliptical ring 1040. In various embodiments, the elliptical ring is not a continuous ring, but is two or more discontinuous ring portions collectively disposed in the shape of a ring. In additional embodiments, the discontinuous ring portions may have different radii and overlap in an alternating fashion, thereby allowing for adaptation to greater variations of astigmatism without compromising the foldability of the lens during implantation.

The elliptical ring 104 may include, consist essentially of, or consist of one or more materials stiffer than (and/or thicker than) that of the lens membrane, e.g., PMMA, acrylic, silicone, and/or parylene polymers, copolymers thereof, and collogen. The elliptical ring 1040 may be molded to (or formed as a portion of) the lens membrane during formation thereof, or the elliptical ring 1040 may be applied to the lens membrane after formation thereof; for example, the elliptical ring 1040 may be coated upon the lens membrane or attached thereto via, e.g., an adhesive. In various embodiments, the elliptical ring 1040 is mostly, or even completely, disposed outside of the optical zone 1010, and thus the elliptical ring 1040 need not be optically transparent.

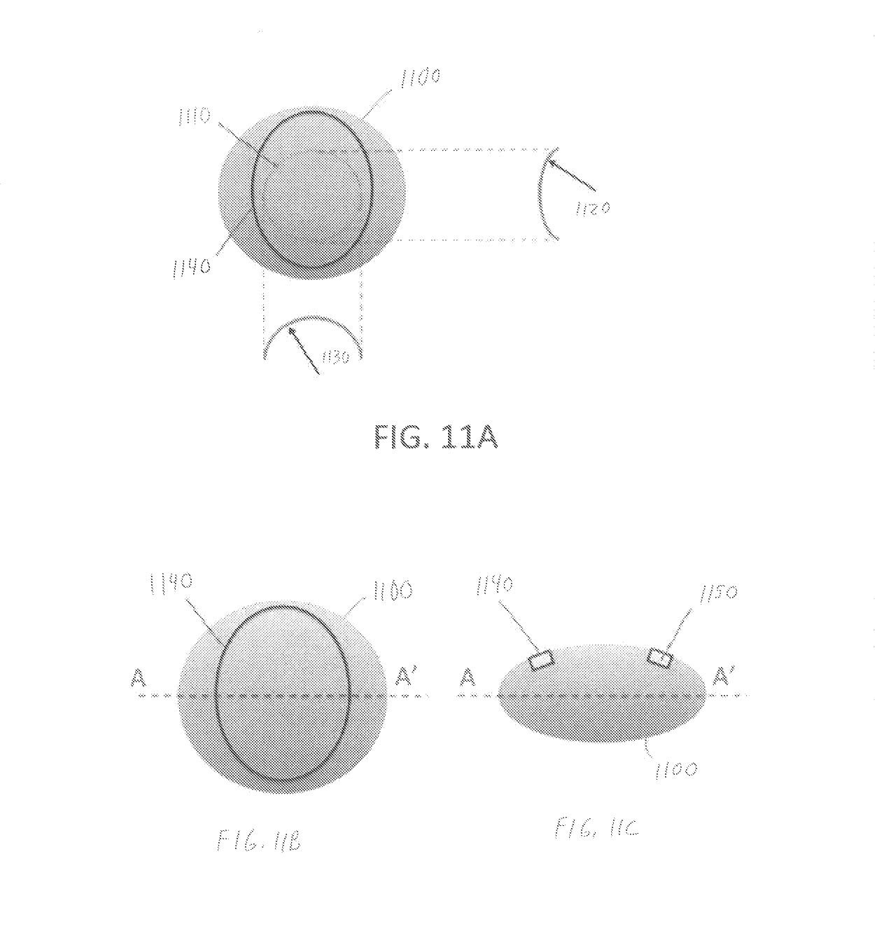

In other embodiments of the invention, an oval profile is introduced into a lens surface via the addition of a hollow ring on the lens surface, as shown in FIG. 11A. FIG. 11A depicts a liquid-filled lens 1100 having an optical zone 1110 (which has radii of curvature 1120, 1130 in perpendicular directions) and a hollow ring 1140 on the lens surface. FIG. 11B depicts a top view of lens 1100, and FIG. 11C depicts a cross-sectional view of lens 1100 though line A-A′. As shown, the hollow ring 1140 may be partially or completely filled with liquid 1150. By filling the ring 1140 with different amounts of liquid, the size (e.g., axes) of the ring 1140 as well as the mechanical strength of the ring 1140 may be adjusted. As described above for elliptical ring 1040, the ring 1140 controllably introduces the designed amount of spherical and cylinder power in the lens, via the adjustable mechanical properties afforded by the amount of liquid fill, thereby enabling the lens 1100 to be toric, and may be made of any of the materials suitable for the elliptical ring 1040.

In some embodiments, one or more fill valves may be used to adjust the surface profile and optical aberration of the lens. Such techniques may be used to reduce total aberrations in the eye system (e.g., to cancel an aberration from the cornea), or to increase specific aberrations for optimal lens performance (e.g., increase spherical aberration to increase depth of field of the lens or create multiple focal points on the anterior surface of the lens). The surface profile of the lens may be adjusted by altering the valve location, valve shape, the locations of multiple valves, and/or the thickness profile of the lens wall. For example, the thickness profile of various portions of the lens wall may be varied with a maximum thickness of the thickest portion of the wall being up to approximately 1000 microns, and preferably under 500 microns. The transition in thickness (or “thickness transition”) between thicker and thinner portions of the wall may be substantially continuously variable (or “smooth”) or abrupt (i.e., approximating a step function between the two thickness values). In various embodiments, the maximum wall thickness in the thickened area is less than or equal to 200 microns.

For the ensuing discussion of the mechanism of creating a custom optical profile, a coordinate system of the lens is hereby defined. The x-axis of the lens is defined as orthogonal to the optical axis (the z-axis) of the lens. The y-axis is orthogonal to both the x-axis and the z-axis. In this coordinate system, the x, y, and z axes are all orthogonal.

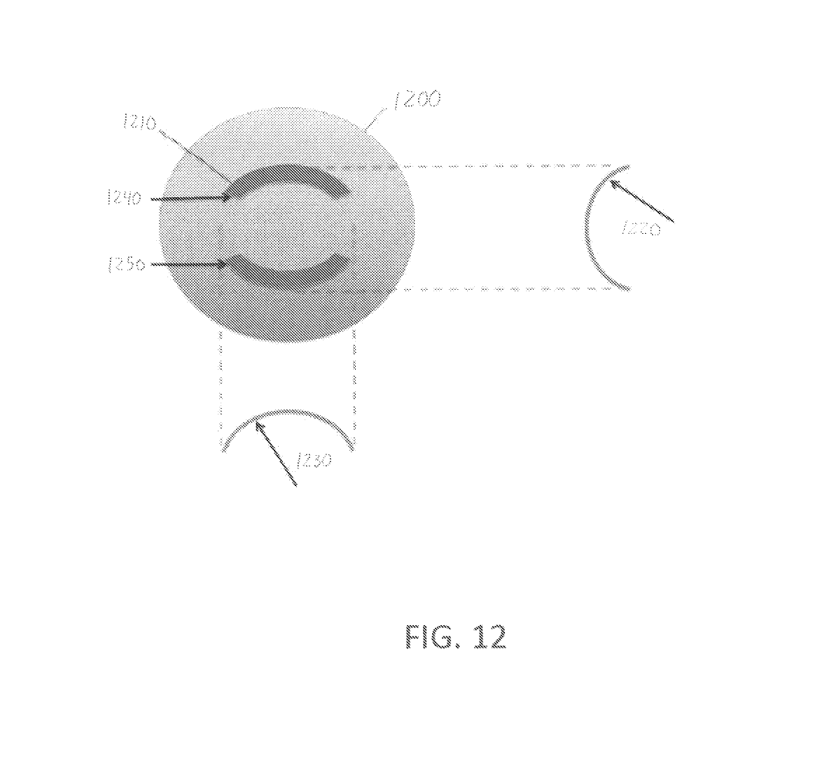

In various embodiments of the invention, a valve portion of the lens causes a desired aberration in the lens. By providing the valve an appropriate dimension, size, and/or location, the lens may be made toric. FIG. 12 depicts an exemplary liquid-filled lens 1200 having an optical zone 1210 and a surface with radii of curvature 1220, 1230. As shown, lens 1200 features two valves 1240, 1250 in or near the optical zone 1210, although embodiments of the invention may include more than two fill valves. The valves 1240, 1250 may be utilized to create a toric shape in which radii of curvature 1220, 1230 are different. In some embodiments, the valves 1240, 1250 are slit valves. Unlike a septum in which each piercing creates a new hole and may irreparably damage a valve, a blunt needle will reversibly open the slit as the needle enters. An elastomeric polymer such as silicone temporarily seals around the needle as the fluid level is adjusted.

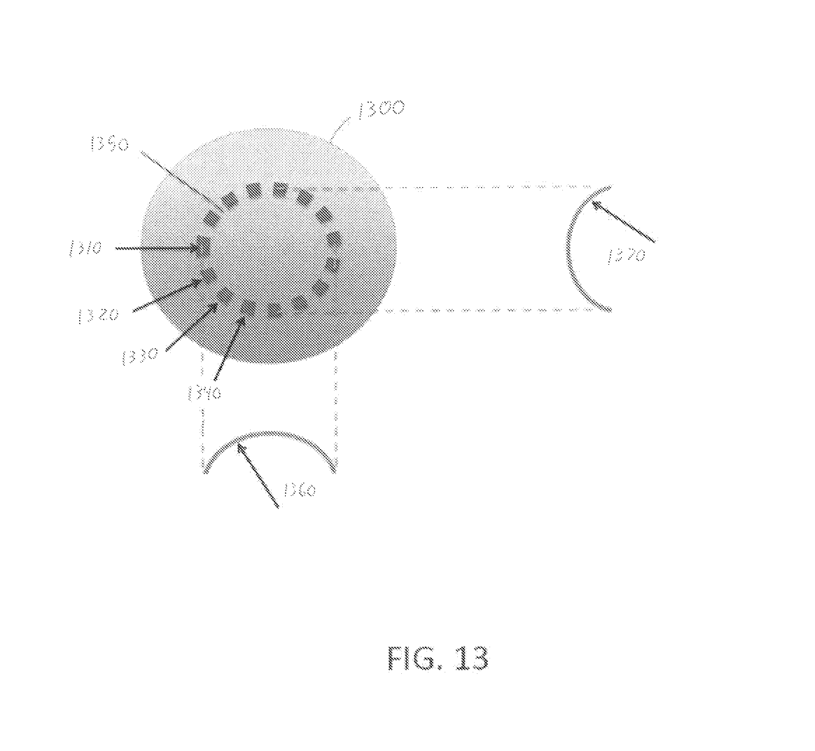

In the embodiment of FIG. 12, the two valves 1240, 1250 are disposed along the x-axis and on opposite sides of the optical axis of the lens 1200, and therefore make the lens mechanically stiffer along the x-axis than along the y-axis. When the lens 1200 is inflated, the y-axis will thus deform more. In this manner, the valve position may be utilized to induce astigmatism in the lens 1200. (In various embodiments, one or more of the valves are slit valves, and disposing the valve along a particular axis makes the lens less stiff along that axis, as the slit in the valve makes the region of the valve more easily deformable.) In various embodiments, this technique is used to create a toric surface on the anterior and/or posterior surfaces of the lens. There may be multiple valves placed in a full or partial concentric pattern to induce the same astigmatism but allow for a more flexible IOL to allow for a smaller incision for insertion. (For example, multiple valves may be disposed on the lens in a pattern such as that of the wall portions depicted in FIG. 13.) The valve(s) need not have a straight wall. In some embodiments, the valve thickness tapers in a direction toward the edge of the lens. This tapering (or chamfer or fillet/round) may allow for a more continuous change in curvature close to the valve. In various embodiments of the invention, the magnitude of the toric shape of the lens is a function of lens fill amount, lens filling fluid characteristics, valve distance from the center of the optical axis, the size and stiffness of the valve, the configuration of the valve(s), and/or configuration of any additional structures in or on the lens.

In various embodiments, the wall thickness or wall stiffness profile of the anterior or posterior portion of the lens is altered to induce an aberration in the lens. As an example, a thickened linear section along the x-axis of the lens on the anterior portion of the lens causes it to be stiffer along the x-axis than the y-axis of the lens. This may be used to create an aberration or a toric shape as described previously. The thickened profile need not be a stepped section. In an embodiment, for a toric shape the wall thickness profile smoothly transitions from thin to thick in a manner that enables the optimum shape of the lens surface without sharp discontinuities. That is, the transition in thickness may be substantially continuously variable or abrupt. The appropriate profile for the desired optical outcome may be modeled by using simulated or empirical analysis, such as finite element analysis, or experimental analysis coupled with an optical analysis. This includes modeling for the appropriate Zernicke coefficient, or aberrational profile of the lens. In other embodiments, an annulus, or capped section of the lens is used to create the appropriate profile. Additional factors such as astigmatism caused by surgical incision size may be factored in to model the appropriate desired optical outcome.

In some embodiments, the desired outcome may be an accommodating IOL with a flexible range of astigmatism that includes the modeled desired optical outcome. The magnitude of the toric shape is a function of the above-mentioned lens properties, but is adjustable after selection and implantation of the lens by modifying the lens filling fluid and volume. Alternatively or in addition, the filling fluid characteristics (e.g. viscosity, composition, etc.) and filling volume (e.g. moderately overfilled) may be tailored to create a non-accommodating IOL, therefore a temporary toric monofocal IOL.

Various embodiments of the invention achieve similar results via portions of the lens being composed of a stiffer material along an axis of the lens, or being coated with a stiffer material, such as parylene. Stiffness may be adjusted by altering total thickness and/or Young's modulus of all or a portion of the lens wall and/or material coated thereon. In various embodiments, two or more materials are used that each have different thicknesses and/or moduli of elasticity. The coating may have different thicknesses at different positions of the lens and/or may form a discrete pattern along the lens. In various embodiments, the lens wall is composed of more than one material, these materials having different mechanical properties (e.g. Young's modulus, Poisson ratio, density, permeability, yield strength, and/or ultimate elongation). FIG. 13 depicts a liquid-filled lens 1300 having a pattern of individual wall portions 1310, 1320, 1330, 1340 for altering lens shape and/or achieving a toric shape of lens 1300. As shown, the wall portions 1310-1340 may be disposed or integrated in or near an optical zone 1350 of lens 1300 and may be utilized to vary either or both of radii of curvature 1360, 1370. As detailed here, the wall portions 1310-1340 may each have different sizes, thicknesses, coatings (and/or coating thicknesses), and/or include or consist essentially of different materials (e.g., materials having different mechanical properties such as stiffness). In this manner, lens 1300 may be customized to achieve a desired astigmatism and/or be adjustable (via, e.g., fluid fill amount and/or fluid fill type) to achieve multiple conformations within a patient's eye.

In various embodiments, a profile of the anterior or posterior section of the lens has a specific lens thickness in order to induce or correct an aberration. In other embodiments, the anterior or posterior surfaces of the lens are manufactured to have multifocal elements, diffractive elements, and/or apodized elements. These elements may be rotationally symmetric around the optical axis of the lens. Stepped profiles may be used, with one side made with a multifocal optic such as a diffractive, or apodized optic. Multifocal optics may have different percentages of near or far light energy transmission based on the pupil size of the patient.

When elements are not rotationally symmetric they may need to be located in a specific orientation relative to the eye. Therefore, one or more alignment/orientation markers on the lens allow angular identification of the lens (e.g., relative to the x and y axis). This allows the lens to be implanted and rotated into the correct location. In various embodiments of the invention, one or more fill valves are used as markers to indicate the angular position of the lens. Alternatively, the alignment markers may be located on the peripheral edge of the lens or any haptic portion of the lens. The lens is implanted and rotated so that the valve(s) and lens are in the appropriate angular position. Or, the lens may be combined with an astigmatism-reducing optical element such as a toric surface. The alignment markers may include, consist essentially of, or consist of, e.g., biocompatible ink or another biocompatible material.

In various embodiments, a fill valve remains in a constant position relative to the eye, and any angular optical corrections on the lens are made relative to the valve. In this manner, the surgeon chooses the appropriate angular correction for each patient. The lens is implanted, and the valve remains in the same position for each patient. As an example, for a toric lens, the appropriate angle used to correct astigmatism is made relative to the valve for each specific lens. Therefore, one lens may correct 1 diopter of astigmatism with an axis at 10 degrees from the lens, while another may correct 1 diopter at an axis 45 degrees from the valve. The surgeon may allow for slight rotation of the lens after implantation for perfect alignment, but rotation may be limited in either direction. In various embodiments, the rotation may be limited to less than ±100 degrees (i.e., 100 degrees in each direction). This may allow surgical access to the valve because the valve is close to the incision site. In addition, by allowing some rotational motion of the valve, the number of lens designs may be reduced, allowing for inventory management efficiencies.

The single-lens, dual-lens, or three-lens systems of AIOLs described herein have been shown for illustrative purposes; embodiments of the present invention are not limited to any particular number of the lenses employed in the implantable AIOL. Any lens systems suitable for use in the AIOLs are within the scope of the present invention.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. An implantable intraocular lens for counteracting an astigmatism aberration of a patient's eye, the lens comprising:
an outer shell defining therewithin an internal space at least partially fillable with a first fluid; and
a toric element disposed on or defined by the outer shell,
wherein, when the outer shell is at least partially filled with the first fluid, the toric element shapes at least a portion of the outer shell to counteract the astigmatism aberration.

2. The lens of claim 1, wherein the toric element comprises a ring disposed on the outer shell.

3. The lens of claim 2, wherein the ring is substantially elliptical.

4. The lens of claim 2, wherein the ring is hollow and at least partially fillable with a second fluid.

5. The lens of claim 2, wherein the ring comprises two or more discontinuous ring portions.

6. The lens of claim 1, wherein the toric element comprises one or more valves extending through the outer shell for selective passage therethrough of the first fluid.

7. The lens of claim 6, wherein the one or more valves are disposed concentrically around an optical zone of the lens.

8. The lens of claim 1, wherein (i) at least a first portion of the outer shell has a first thickness, and (ii) the toric element comprises one or more second portions of the outer shell having a second thickness greater than the first thickness.

9. The lens of claim 8, wherein a thickness transition between a first portion of the outer shell and a second portion of the outer shell is substantially continuous.

10. The lens of claim 8, wherein a thickness transition between a first portion of the outer shell and a second portion of the outer shell is abrupt.

11. The lens of claim 1, wherein (i) at least a first portion of the outer shell has a first stiffness, and (ii) the toric element comprises one or more second portions of the outer shell having a second stiffness greater than the first stiffness.

12. The lens of claim 1, wherein the toric element comprises one or more portions of the outer shell having a coating disposed thereon.

13. The lens of claim 12, wherein the coating comprises parylene.

14. The lens of claim 1, further comprising one or more alignment markings disposed on the outer shell.

15. The lens of claim 1, wherein a degree of filing of the outer shell with the first fluid determines, at least in part, one or more optical properties of the lens.

16. The lens of claim 1, wherein the outer shell is accommodating.

17. The lens of claim 1, wherein the outer shell is non-accommodating.

18. The lens of claim 1, further comprising an internal optic at least partially within the outer shell, the internal optic comprising one or more toric elements.

19. The lens of claim 1, wherein the outer shell includes at least one of a multi-focal element, a diffractive element, or an apodized element.

20. A method for counteracting an astigmatism aberration of a patient's eye, the method comprising:
implanting within the patient's eye an intraocular lens, the intraocular lens having an outer shell and a toric element disposed on or defined by the outer shell; and
at least partially filling the lens with a first fluid, whereby the toric element shapes at least a portion of the outer shell to counteract the astigmatism aberration.

21. The method of claim 20, wherein the toric element comprises a ring disposed on the outer shell.

22. The method of claim 21, further comprising at least partially filling at least a portion of the ring with a second fluid.

23. The method of claim 20, wherein the toric element comprises one or more valves, the lens being at least partially filed with the first fluid via the one or more valves.

24. The method of claim 20, further comprising aligning the lens within the patient's eye via one or more alignment markings disposed on the outer shell.
